(19) United States
(12) Patent Application Publication
(10) Pub. No.: US 2005/0154372 A1
(43) Pub. Date: Jul. 14, 2005

(54) Needleless injection adapter

(75) Inventor: Susumu Minezaki, Tokyo (JP)

Correspondence Address: STAAS & HALSEY LLP, SUITE 700, 1201 NEW YORK AVENUE, N.W., WASHINGTON, DC, 20005, US

(73) Assignee: Orchis Company Ltd., Tokyo (JP)

(21) Appl. No.: 10/930,837

(22) Filed: Sep. 1, 2004

(30) Foreign Application Priority Data
Jan. 14, 2004 (JP) 2004-007348

(51) Int. Cl. A61M 25/16

(52) U.S. Cl. 604/533

(57) ABSTRACT

The invention provides a needleless injection adapter which can inject medical fluid without using an injection needle, and can completely prevent occurrence of accidental needle sticking to medical staff and a patient and of accidental nosocomial infection. The needleless injection adapter includes: a socket being coupled to a tip of an injector; a duct body with connecting portions to connect with fluid conveying tubes at both ends thereof; a movable member with the socket, slidably held on the middle of a surface of the duct body. A first hole extending to the socket and a second hole extending through the duct body are opposed to each other on a face where the movable member and the duct body are slidable in contact with each other. The two holes are connected or disconnected in accordance with the position of the movable member.

### CROSS REFERENCE TO RELATED APPLICATION

This application is based upon and claims the benefit of priority from Japanese Patent Application No. 2004-007348, filed on Jan. 14, 2004, the entire contents of which are incorporated herein by reference.

### BACKGROUND OF THE INVENTION

1. Field of the Invention

The present invention relates to a needleless injection adapter which is disposed in the middle of an extracorporeal blood circuit used for treatment for renal failure and the like, or with a blood or fluid transfusion set, in order to inject medical fluid into a patient or collect blood from a patient with the extracorporeal blood circuit at appropriate time.

2. Description of the Related Art

A conventional injection adapter is generally provided in the middle of an extracorporeal blood circuit used for treatment for a patient of renal failure, and in the middle of a blood or fluid transfusion set. The conventional injection adaptor, as shown in FIG. 13, is provided with a duct body 73 having connecting portions 72 to connect with the fluid conveying tubes 71 at the ends thereof. An erect duct 74 is provided in the middle of the top face of the duct body 73. A cap 76 is formed in the top end of the erect duct 74, holding a rubber plug 75 pierced with an injection needle 80b in such a manner as to expose a part of the rubber plug 75.

In the conventional injection adapter, the rubber plug 75 exposed from the cap 76 is pierced with the injection needle 80b coupled to a tip 80a of an injector 80 at its base end. Medical fluid is injected by pushing a plunger as indicated by the arrow. Upon pulling the injection needle 80b out of the rubber plug 75 after the injection of the medical fluid, a hole of the needle is immediately clogged by the elasticity of the rubber. Thus, the injection operation of the medical fluid does not cause any trouble over original treatment operation, in which a patient is connected to an extracorporeal blood circuit or a blood or fluid transfusion set.

After the medical fluid injection, the injection needle is pulled out of the rubber plug 75 as described above, and a protective cap (not illustrated) is immediately put thereon, and the injection needle with the protective cap thereon is detached from the injector from segregated discarding. However, there is a possibility that an operator may accidentally stick the tip of the injection needle into his/her finger when putting the injection needle on the protective cap, and may get infected from infectious blood adhering to the needle.

### SUMMARY OF THE INVENTION

In view of solving the above-identified problems, an object of the present invention is to provide a needleless injection adapter which can inject medical fluid without use of an injection needle and completely prevents occurrence of accidental needle sticking into medical staff and a patient and of accidental nosocomial infection.

To achieve the foregoing object, a needleless injection adapter according to one aspect of the present invention includes a duct body, a socket, a movable member, and biasing member. The duct body has connecting portions to connect with fluid conveying tubes at both ends thereof. The movable member having the socket, to which a tip of an injector is coupled, is slidably held in the middle of a surface of the duct body. Two holes, one extending to the socket and the other one extending through the duct body, are disposed oppositely to each other, in a surface on which the movable member and the duct body are slidably in contact with each other. The biasing member biases the movable member in a direction where the two holes are misaligned with each other. With the tip of the injector without a needle coupled to the socket, a movement of the movable member connects the two holes to carry out injection. After the injection, the movable member is automatically displaced to misalign the holes.

According to the needleless injection adapter of the present invention, it is possible to carry out injection by coupling the tip of the injector without a needle to the socket and moving the movable member to connect the holes. It is also possible to misalign the holes by automatically displacing the movable member after the injection. Therefore, the needleless injection adapter has a beneficial effect of quickly completing the medical fluid injection operation.

In another aspect of the needleless injection adapter according to the present invention, a needleless injection adapter includes a duct body, a socket, a slidable member, and biasing member. The duct body has connecting portions to connect with fluid conveying tubes at both ends thereof, and a vertical hole to connect the socket to the inside of the duct body. The socket, to which the tip of the injector is coupled, is provided in the middle of a surface of the duct body. The slidable member divides the vertical hole, and is provided with at least one connection hole which connects upper and lower vertical holes divided by the slidable member. The biasing member biases the slidable member in a direction where the connection hole shifts from the upper and lower vertical holes. By coupling the tip of the injector without a needle to the socket, and sliding the slidable member, the upper and lower vertical holes are connected to carry out injection. After the injection, the slidable member is automatically displaced to divide the upper and lower vertical holes.

According to the foregoing needleless injection adapter, it is possible to carry out injection by coupling the tip of the injector without a needle to the socket, and sliding the slidable member to connect the upper and lower vertical holes. It is also possible to divide the upper and lower vertical holes by automatically displacing the slidable member after the injection. Therefore, the needleless injection adapter has a beneficial effect of quickly completing the medical fluid injection operation.

In another aspect of the needleless injection adapter according to the present invention, the biasing member is fixed on the duct body so that the biasing member accumulates return force to be elastically deformed in a state that the socket gets in communication with the inside of the duct body by the movement of the movable member or the sliding of the slidable member. When the socket and the inside of the duct body are communicated with each other, the biasing member accumulates the return force. After the injection, releasing the accumulated return force at the position where they are in communication brings the socket and the inside of the duct body into a non-communication state.

This makes it possible for the biasing member to accumulate the return force only by connecting the socket to the inside of the duct body. It is beneficial that the socket is immediately disconnected from the inside of the duct body by the return force of the biasing member after the completion of the injection, without any manipulation of an operator.

In further aspect of the needleless injection adapter according to the present invention, the socket is provided with a stopper which retains the state that the socket is in communication with the inside of the duct body. When the socket gets in communication with the inside of the duct body by moving the movable member or sliding the slidable member, and the tip of the injector is coupled to the socket, the stopper operates to retain the state until it is released.

According to the foregoing needleless injection adapter, moving the movable member or sliding the slidable member to get the socket in communication with the inside of the duct body after the coupling of the tip of the injector to the socket allows the stopper to operate to retain the communication state until the completion of injection without any manipulation of an operator. With the needleless injection adapter connected to another fluid transfusion set and the like, it is beneficial that continuous injection is enabled.

In further aspect of the needleless injection adapter according to the present invention, the needleless injection adapter includes a duct body, a socket, a rotatable member, a spring member, and a sound emission member and/or a tactile member. The rotatable member with the socket for the injector is slidably disposed on a side face of the duct body. When the rotatable member is rotated by a predetermined angle, a small hole extending to the socket of the rotatable member is aligned with another small hole formed in the side face of the duct body. The spring member biases the socket in the direction where the small holes are misaligned with each other. The sound emission member and/or the tactile member provided in the socket snaps a tip end of the spring member at a point where the small holes are aligned, so that it is possible to confirm that the injection is normally done.

This makes it possible to align the small hole extending to the socket of the rotatable member with another small hole extending through the side face of the duct body, by coupling the socket erected on the rotatable member to the tip of the injector without a needle to rotate the rotatable member, whereby the operator is surely informed of the alignment position with a sound or an impact by snapping the tip end of the spring member. Therefore, it is advantageous that the operator is able to smoothly and securely carry out the injection since he or she can operate the injector with the rotatable member in rotation upon hearing the sound or impact. Also, when the operator loosens his/her hold after the injection, the rotatable member is automatically returned to a position in which the small holes are misaligned with each other, due to the action of the spring member. This eliminates the need for the operator to return the rotatable member to the misalignment position of the small holes. This also enables the operator to pull the tip of the injector out of the socket at ease. Accordingly, the needleless injection adapter provides various beneficial effects.

In further aspect of the needleless injection adapter according to the present invention, the sound emission member and/or the tactile member includes the tip end of the spring member whose base end is held by the duct body, and a projection or a recess on the side face of the socket with which the tip end in contact. The sound emission member and/or the tactile member are so structured as to surely emit a sound or an impact at the point where the small holes are aligned.

According to the foregoing needleless injection adapter, the sound or impact is emitted when the tip end of the spring member makes contact with the projection or the recess on the side face of the socket. Thus, it is possible to easily make the emission of the sound or the impact coincide with the alignment of the small holes, so that the operator is beneficially informed of the alignment of the small holes with precision.

In another aspect of the needleless injection adapter according to the present invention, the needleless injection adapter is provided with a position retaining member which retains the rotatable member to rotate against the spring force of the spring member, in such a position that the small holes are aligned with each other. The alignment of the small holes is continuously maintained.

According to the foregoing needleless injection adapter, the position retaining member in the rotatable member can continuously retain the alignment of the small holes during continuous medical fluid injection, if medical fluid is continuous injected for many hours with use of a continuous medical fluid injector coupled to the socket of the rotatable member, for example. Also, without the position retaining member, the action of the spring member automatically misaligns the small holes. Therefore, it is advantageous that the needleless injection adapter is connected to and disconnected from the continuous medical fluid injector at ease.

### BRIEF DESCRIPTION OF THE DRAWINGS

The nature, principle, and utility of the invention will become more apparent from the following detailed description when read in conjunction with the accompanying drawings in which like parts are designated by identical reference numbers, in which:

FIG. 1 is a perspective view of a needleless injection adapter according to a first embodiment of the present invention;

FIG. 2 is a front sectional view of the needleless injection adapter according to the first embodiment, in which biasing member is elastically deformed with accumulated return force;

FIG. 3 is a front sectional view of the needleless injection adapter according to the first embodiment, in which the return force of the biasing member brings a socket and the inside of a duct body into a non-communication state;

FIG. 4 is an enlarged sectional view of the needleless injection adapter with a stopper according to the first embodiment;

FIG. 5 is a perspective view of a needleless injection adapter according to a second embodiment of the present invention;

FIG. 6 is a front sectional view of the needleless injection adapter according to the second embodiment, in which biasing member is elastically deformed with accumulated return force;

FIG. 7 is a front sectional view of the needleless injection adapter according to the second embodiment, in which the return force of the biasing member brings a socket and the inside of a duct body into a non-communication state;

FIG. 8 is an enlarged sectional view of the needleless injection adapter with a stopper according to the second embodiment;

FIG. 9 is a perspective view of a needleless injection adapter according to a third embodiment of the present invention;

FIG. 10 is an enlarged sectional view of the needleless injection adapter according to the third embodiment of the present invention;

FIG. 11 is a sectional view of the needleless injection adapter according to the third embodiment, in which small holes are misaligned with each other;

FIG. 12 is a sectional view of the needleless injection adapter according to the third embodiment, in which the small holes are aligned with each other; and

FIG. 13 is a perspective view showing a conventional injection adapter with an injection needle.

### DESCRIPTION OF THE PREFERRED EMBODIMENTS

The present invention will be hereinafter described on the basis of embodiments shown in drawings.

### First Embodiment

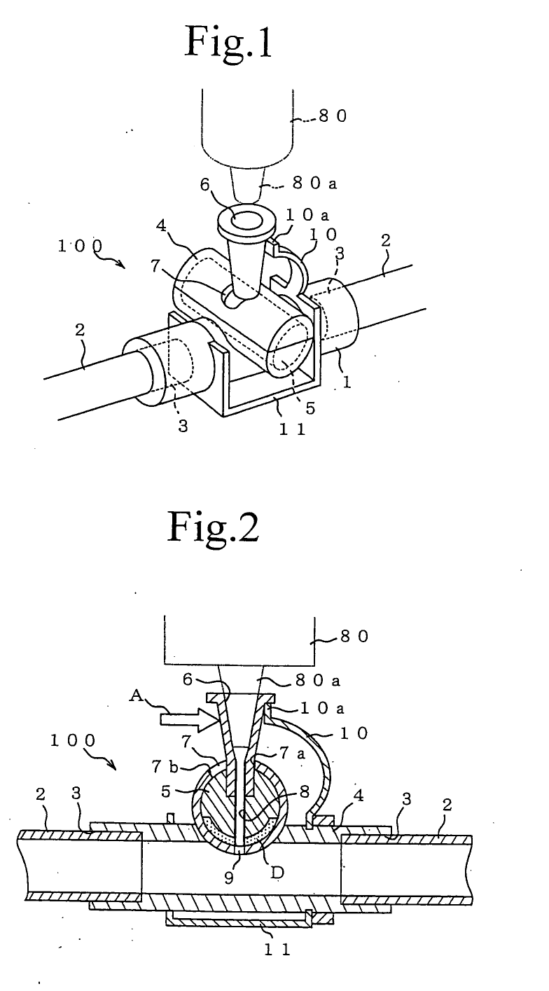

A needleless injection adapter 100 according to a first embodiment of the present invention will be described with reference to FIGS. 1 to 4.

The needleless injection adapter 100 according to the first embodiment includes a duct body 1 having the connecting portions 3 of fluid conveying tubes 2 at both ends thereof, and a tubular member 4 which is integrally superposed on the middle of a surface of the duct body 1. The tubular member 4 is orthogonal to the duct body 1 in FIG. 1, but the tubular member 4 may cross obliquely or in parallel with the duct body 1. The fluid conveying tubes 2 connect a patient to an extracorporeal blood circuit (not illustrated) or a vial of a blood or fluid transfusion set.

A movable member 5 is movably held in the tubular member 4, so that it is slidably in contact with a surface of the duct body 1. The movable member 5 is provided with a socket 6 to which a tip 80a of an injector 80 is coupled. The socket 6, as shown in FIG. 2, is exposed through a guide groove 7 which is provided in the top face of the tubular member 4. The guide groove 7 regulates the moving direction and moving range of the socket 6.

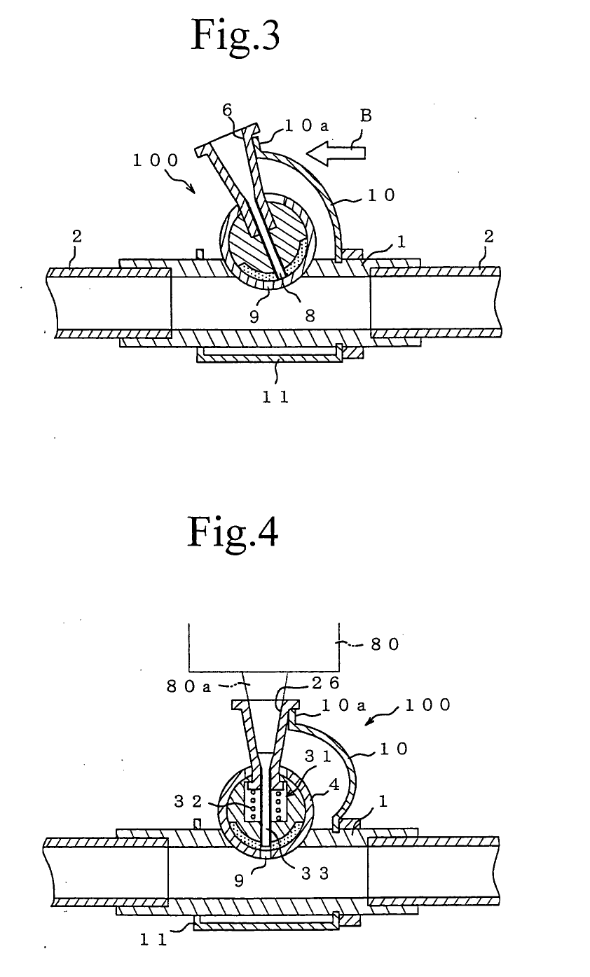

A hole 8 is formed in the bottom face of the movable member 5 (a sliding surface on the duct body 1) and extends to the socket 6, and a hole 9 is formed in the bottom face of the tubular member 4 (a sliding surface on the movable member 5) and extends through the duct body 1. These facing two holes 8 and 9, as shown in FIG. 2, are aligned with each other (a communication state) in a position where the movable member 5 is moved along the guide groove 7. The holes 8 and 9, as shown in FIG. 3, are misaligned with each other (a non-communication state) in another position.

To connect or not to connect the two holes 8 and 9 in a highly fluid-tight manner, as shown in the drawings, elastic material (soft plastic material or rubber material) D with a hole corresponding with the hole 8 may be fitted in the bottom face of the movable member 5. Elastic material (not illustrated) may be fitted on the bottom face of the tubular member 4, as a matter of course.

In this embodiment, the two holes 8 and 9 are in the communication state when the socket 6 of the movable member 5 is erect with making contact with a groove edge 7a of the guide groove 7, and are in the non-communication state when the socket 6 of the movable member 5 is oblique with making contact with the other groove edge 7b of the guide groove 7. The two holes 8 and 9, however, may be in the communication state when the socket 6 is oblique, and may be in the non-communication state when the socket 6 is erect.

The moving direction and range of the movable member 5 is in the circumferential direction of the tubular member 4 in an example shown in the drawing. The movable member 5 may slide inside the tubular member 4 in an axial direction, or in the circumferential direction in combination with the axial direction.

The socket 6 is biased by biasing member 10 in such a direction that the hole 8 of the movable member 5 is misaligned with the hole 9 of the duct body 1. The biasing member 10 extends from an end portion of a fixing member 11 whose cross section has the shape of “U”. The fixing member 111 fixes the duct body 1 by which pinching it with the tubular member 4. An extended end 10a of the biasing member 10 makes contact with a surface of the socket 6. When pushing force as shown by the arrow A of FIG. 2 is applied to the socket 6 to align both holes 8 and 9 with each other, the biasing member 10 is elastically deformed with accumulated return force. When the pushing force is loosened, on the other hand, the movable member 5 obliquely moves by the return force (the arrow B) of the biasing member 10, as shown in FIG. 3, to bring both holes 8 and 9 back into the non-communication state.

The socket 6 or 26 is provided with a stopper 31 which keeps the communication between the socket 6 or 26 and the inside of the duct body 1 or 21 (refer to FIG. 4). The stopper 31 prevents the communication between the hole 8 extending to the socket 26 and the hole 9 extending through the duct body 1 from being released before the completion of injection.

According to this embodiment, the stopper 31 is so structured that the socket 26 can be pushed in a downward direction against the spring force of a spring 32 while being coupled to the tip 80a of the injector 80. Thus, a slider 33 integral with the socket 26 is inserted into the hole 9 formed in the bottom face of the tubular member 4. This insertion is retained until the completion of injection, in which the tip 80a of the injector 80 is pulled out of the socket 26 by the pushing force of the biasing member 10.

In the following, the operation of the needleless injection adapter 100 according to the first embodiment will be described.

First, the needleless injection adapter 100 according to this embodiment is disposed with the fluid conveying tubes 2 of an extracorporeal blood circuit (not illustrated). The socket 6 of the movable member 5 is oblique at first, as shown in FIG. 3, so that the hole 8 extending to the socket 6 and the hole 9 extending through the duct body 1 are in the non-communication state to carry out dialysis treatment and the like.

When it is necessary to inject medical fluid into a patient, the tip 80a of the injector 80 containing the medical fluid is coupled to the socket 6 or 26. Then, since the socket 6 is moved along the guide groove 7 with the injector 80, the two holes 8 and 9 are brought into the communication state against the biasing member 10. An operator has to manually keep the communication during the injection, to prevent the return force of the biasing member 10. With the stopper 31 in the socket 26, however, it is possible to retain the communication without the manipulation of the operator.

Then, pushing a plunger of the injector 80 makes it possible to inject the medical fluid into blood (transfused fluid) which flows through the fluid conveying tubes. In completing this injection operation, the tip 80a of the injector 80 is pulled out of the socket 6, so that the return force of the biasing member 10 brings the two holes 8 and 9 into the non-communication state.

This completes a series of medical fluid injection operation without any effect given on original treatment operation in which the patient is connected to the extracorporeal blood circuit (the blood or fluid transfusion set). As a matter of course, the needleless injection adapter according to this embodiment is applicable to blood collection (suction into the injector), in addition to the injection of the medical fluid.

The stopper 31 is disposed inside the socket 26 according to the foregoing embodiment but it may be disposed in another place. The duct body 1 may be provided with, for example, a lever for regulating the return force of the biasing member 10 which is elastically deformed by accumulating the return force.

### Second Embodiment

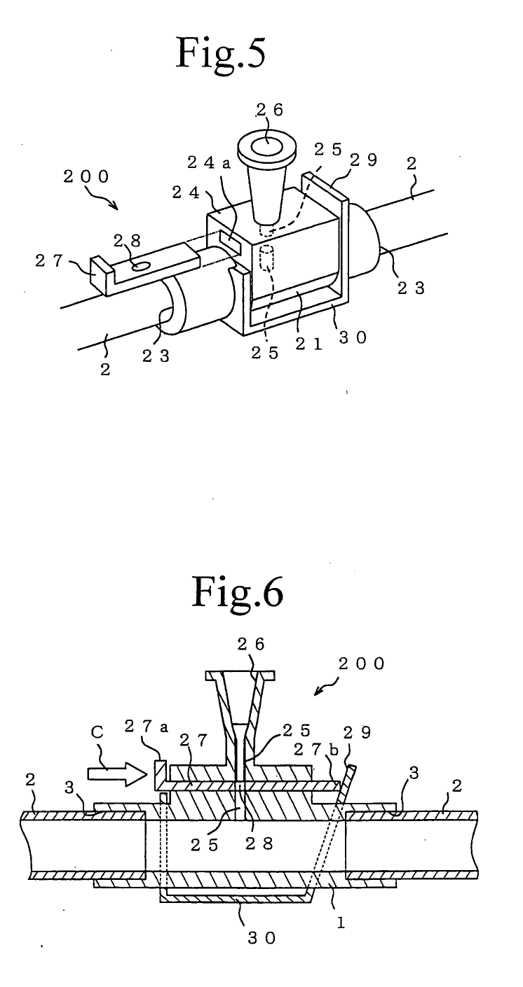

A needleless injection adapter 200 according to a second embodiment of the present invention will be described with reference to FIGS. 5 to 8.

The needleless injection adapter 200 according to the second embodiment is provided with a duct body 21 having connecting portions 23 of the fluid conveying tubes 2 at both ends thereof, and a cabinet 24 which is integrally formed on a surface in the middle of the duct body 21. In FIG. 5, the cabinet 24 is provided around the perimeter of the duct body 21.

A socket 26, to which the tip 80a of the injector 80 is coupled, is provided in the top face of the cabinet 24. The socket 26 is connected to the inside of the duct body 21 through a vertical hole 25. The vertical hole 25 is vertically divided by a slidable member 27 which is slidably inserted into a horizontal hole 24a penetrating both ends of the cabinet 24. A connection hole 28 is formed in the slidable member 27 to connect the divided upper and lower vertical holes 25. The number of the connection hole 28 is one in the drawing but it is not limited thereto. The connection hole 28 is a perfect circle in shape in general, but it does not always have to be the perfect circle.

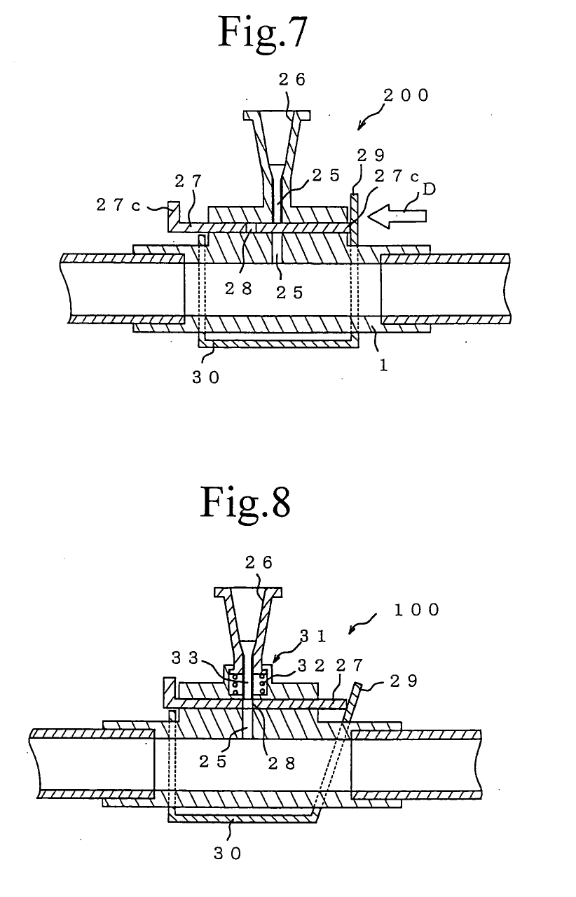

In sliding the slidable member 27 as shown by the arrow C of FIG. 6, the connection hole 28 in the slidable member 27 is aligned with the vertical hole 25 (communication state) which connects the socket 26 to the inside of the duct body 21. In sliding the slidable member 27 as shown by the arrow D of FIG. 7, the connection hole 28 shifts from the vertical hole 25 (non-communication state).

The vicinity of the connection hole 28 of the slidable member 27 or the whole of the slidable member 27 may be made of elastic material (soft plastic material or rubber material), in order to connect or not to connect the connection hole 28 to the vertical hole 25 in a highly fluid-tight manner. The edge of the vertical hole 25 may be made of elastic material (not illustrated).

One end of the slidable member 27 is provided with a handle 27a, and the other end 27b thereof slightly protrudes from the horizontal hole 24a. The protruding end 27b makes contact with biasing member 29 which biases the slidable member 27 so as to shift the connection hole 28 of the slidable member 27 from the vertical hole 25. The biasing member 29 extends from an end portion of a fixing member 30 with a U-shaped cross section which fixes the cabinet 24 by pinching it with the duct body 21. When the handle 27a provided in one end of the slidable member 27 is pressed as shown by the arrow C of FIG. 6, to align the hole 28 with the vertical hole 25, the biasing member 29 is elastically deformed with accumulated return force. In loosening the pressing force, on the other hand, the biasing member 29 slides the slidable member 27 by the return force (the arrow D) as shown in FIG. 7, so that both the holes 28 and 25 are brought into the non-communication state.

The socket 26 is provided with a stopper 31 which keeps the communication between the socket 26 and the inside of the duct body 21 (refer to FIG. 8). The stopper 31 prevents the communication between the connection hole 28 of the slidable member 27 and the vertical hole 25 from being released before the completion of injection.

The stopper 31 is structured so that the socket 26 presses a spring 32 in a downward direction against the spring force of the spring 32 while coupled to the tip 80a of the injector 80. Thus, a slider 33 integral with the socket 26 is inserted into the hole 28 of the slidable member 27. This insertion is retained until the completion of injection, in which the tip 80a of the injector 80 is pulled out of the socket 26 by the pressing force of the biasing member 29.

The operation of the needleless injection adapter 200 according to the second embodiment will be described in the following.

First, the needleless injection adapter 100 or 200 according to the first or second embodiment is disposed in the middle of the fluid conveying tubes 2 of an extracorporeal blood circuit (not illustrated). With the needleless injection adapter 100 according to the first embodiment, the socket 6 of the movable member 5 is oblique at first as shown in FIG. 3, so that the hole 8 extending to the socket 6 and the hole 9 extending through the duct body 1 are in the non-communication state. With the needleless injection adapter 200 according to the second embodiment, the slidable member 27 is in an initial position, so that the hole 28 is not in communication with the vertical hole 25 for connecting the socket 26 to the inside of the duct body 21. Dialysis treatment and the like are carried out in this state.

When it is necessary to inject medical fluid into a patient, the tip 80a of the injector 80 containing the medical fluid is coupled to the socket 6 or 26. Then, since the slidable member 27 is slid with the injector 80, the two holes 28 and 25 are brought into the communication state against the biasing member 29. An operator has to manually keep the communication during the injection operation, to prevent the return force of the biasing member 29. With the stopper 31 provided in the socket 26, however, it is possible to retain the communication state without the manipulation of the operator.

Then, pushing the plunger of the injector 80 makes it possible to inject the medical fluid into blood (transfused fluid) which flows through the fluid conveying tubes. In completing this injection operation, the tip 80a of the injector 80 is pulled out of the socket 26, so that the return force of the biasing member 29 brings the two holes 28 and 25 into the non-communication state. This completes a series of injection operation of the medical fluid without any effect given on original treatment operation in which the patient is connected to the extracorporeal blood circuit (the blood or fluid transfusion set). As a matter of course, the needleless injection adapter according to this embodiment is applicable to blood collection (suction into the injector), in addition to the injection of the medical fluid.

The stopper 31 is disposed inside the socket 26 according to the foregoing embodiment, but may be disposed in another position. The duct body 21 may be provided with, for example, a lever for regulating the return force of the biasing member 29 which is elastically deformed with accumulated return force.

### Third Embodiment

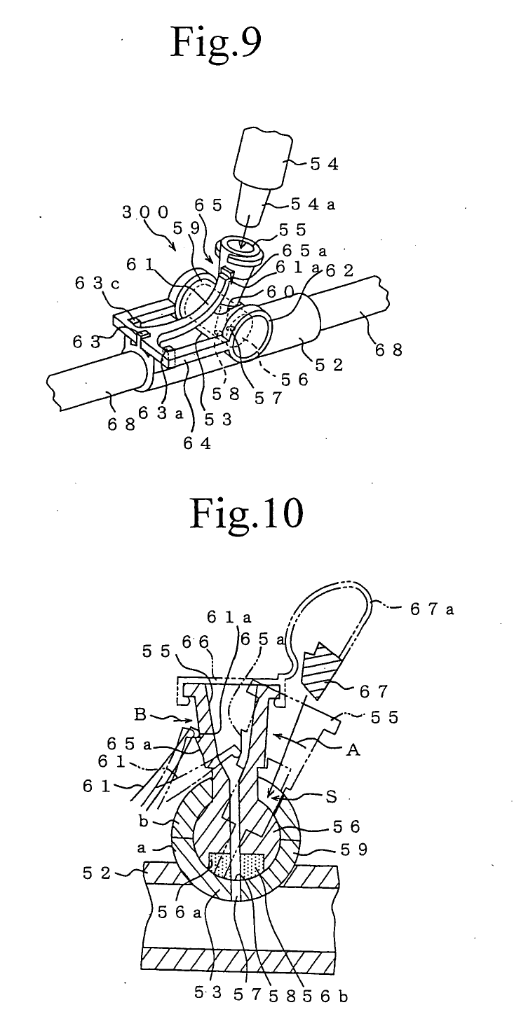

A needleless injection adapter 300 according to a third embodiment of the present invention will be described with reference to FIGS. 9 to 12.

In the needleless injection adapter 300 according to the third embodiment, a rotatable member 56 having a socket 55, to which a tip 54a of an injector 54 is coupled, is slidably disposed in a side face 53 of a duct body 52. When the rotatable member 56 is rotated by a predetermined angle (for example, the rotatable member 56 becomes orthogonal to the duct body 52), a small hole 58 extending to the socket 55 of the rotatable member 56 is aligned with a small hole 57 which is formed in the side face 53 of the duct body 52 to extend through the duct.

The rotatable member 56 refers to what is rotatably contained in a duct 59 which is fixed over the duct body 52 in an orthogonal manner. Thus, the side face 53 of the duct body 52 corresponds to the bottom face of the orthogonal duct 59. The small hole 57 is formed at one position (in the center) on the bottom face of the orthogonal duct 59, and extends through the side face 53 of the duct body 52. Furthermore, the socket 55 erected on the rotatable member 56 protrudes to outside from a through hole 60 which is formed in the top wall of the orthogonal duct 59 along its circumference.

To secure the air tightness between the small hole 57 extending through the side face 53 of the duct body 52 and the small hole 58 extending to the socket 55 of the rotatable member 56, a packing member 56b may be fitted into a notch groove 56a formed in the bottom face of the rotatable member 56, and the small hole 58 may extend and penetrate the packing member 56b. In this case, if necessary, means may be provided for preventing the leakage of fluid from a contact surface between the small hole 58 in the rotatable member 56 and the small hole 58 in the packing member 56b, such as a projection (not illustrated) in the shape of an O-ring to surround the periphery of the small hole 58 in the rotatable member 56. The projection in the shape of the O-ring may also surround the periphery of the small hole 57 in the duct body 52, to function as leakage prevention means during the alignment of the small hole 57 with the small hole 58 in the packing member 56b.

The side face of the socket 55 erected on the rotatable member 56 is biased and inclined by the tip end 61a of a spring member 61 whose base end is held by the duct body 52. Thus, the small holes 57 and 58 are misaligned from each other in a normal state (refer to FIG. 10). The base end of the spring member 61 extends from ring-shaped members 62 fitted over both end portions of the orthogonal duct body 59. The base end of the spring member 61 is integral with a base frame 64 supported by pins 63, 63a, and 63b which stand near an end of the duct body 52. The ring-shaped members 62 integral with the base frame 64 are conducive to the coalescence between the divided two portions of the orthogonal duct 59, the bottom face portion a and the top face portion b.

The socket 55 of the rotatable member 56 is provided with a sound emission member (and/or tactile member) 6 which snaps the tip end 61a of the spring member 61 to emit a sound or an impact at an alignment position of the small holes 57 and 58. The sound emission member 65 is composed of the tip end 61a of the spring member 61 whose base end is held by the duct body 52, and a projection 65a provided on the side face of the socket 55 making contact with the tip end 61a. Namely, the tip 54a of the injector 54 without a needle is coupled to the socket 55 erected on the rotatable member 56 to rotate the rotatable member 56 with the injector 54, thereby aligning the small holes 57 and 58 with each other. At this time, held by the duct body 52 at the base end, the tip end 61a of the spring member 61 falls down from the top of the projection 65a provided on the side face of the socket 55, and is snapped down to tap the side face of the socket 55, emitting a sound or an impact.

In other words, when the socket 55 with the injector 54 coupled thereto is rotated from a position illustrated by chain double-dashed lines to a position illustrated by solid lines in the direction of the arrow A in FIG. 10, the tip end 61a of the spring member 61 runs up on a projection 65a and falls down from the top portion thereof in the direction of the arrow B. The tip end 61a of the spring member 61 is snapped by the projection 65a to tap the side face of the socket 55, emitting a sound (or an impact). Instead of the projection 65a, a recess (not illustrated) may be provided in the side face of the socket 55. In this case, the tip end 61a of the spring member 61 falls down into the recess from the end portion of the recess and is snapped.

The socket 55 of the rotatable member 56 of the needleless injection adapter 300 according to the third embodiment may be provided with a cap 66 which is put on the socket 55 while the needleless injection adapter 300 is in no use. The cap 66 is used as necessary for preventing invasion of bacteria, dust, and the like into the socket 55. As a matter of convenience, it is preferably configured such that sterilizing fluid can flow through the socket 55 with the cap 66 put on during the sterilization of the needleless injection adapter 300 according to the third embodiment, and after the sterilization the socket 55 is completely sealed by, for example, strongly pressing the cap 66 into the socket 55.

Being coupled to a continuous medical fluid injector (not illustrated), the needleless injection adapter 300 according to the third embodiment may be used for continuous medical fluid injection of long hours. In this case, it is necessary to retain an alignment between the small holes 57 and 58 by rotating the rotatable member 56 against the spring force of the spring member 61. The needleless injection adapter 300 according to the third embodiment is provided with a position retaining member 67, as an accessory for this purpose, which rotates the rotatable member 56 against the spring force of the spring member 61 to retain an alignment position of the small holes 57 and 58. The structure of the position retaining member 67 is not specifically limited as long as the position retaining member 67 can retain the rotational position of the rotatable member 56 while the small holes 57 and 58 are aligned with each other. In this embodiment, the position retaining member 67 is a wedge-shaped member shown by chain double-dashed lines which is inserted into the clearance S occurring between the duct body 52 and the rotatable member 56 when the small holes 57 and 58 are aligned with each other. Needless to say, the wedge-shaped member has to be configured not to be easily pulled out from the clearance S when once inserted thereinto, except that the operator pulls it out on purpose.

In this embodiment, as shown in FIG. 10, the wedge-shaped member as an example of the position retaining member 67 is formed in the end of a line member 67a extending from the cap 66 put on the socket 55 of the rotatable member 56. The wedge-shaped member, however, may be formed in another member, for example, in a line member (not illustrated) extending from the base frame 64 of the spring member 61. The wedge-shaped member may be independent from the cap 66.

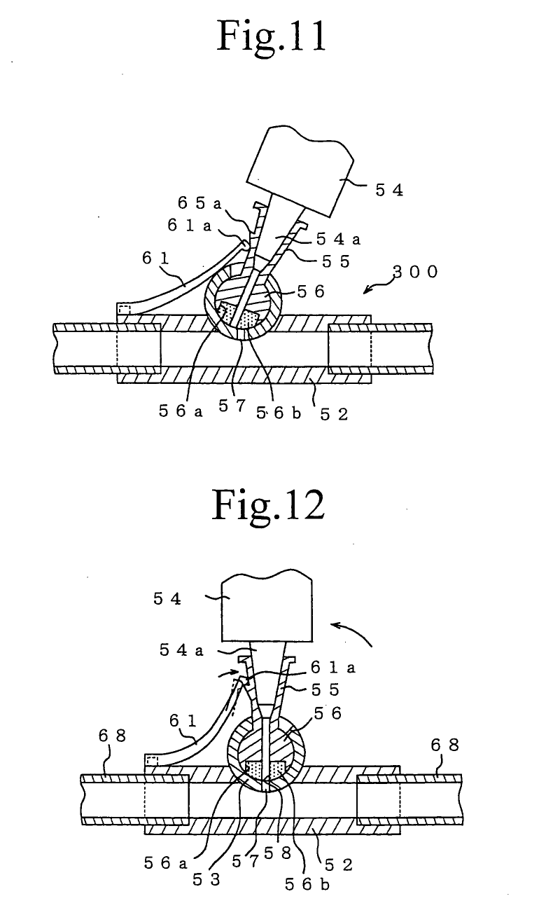

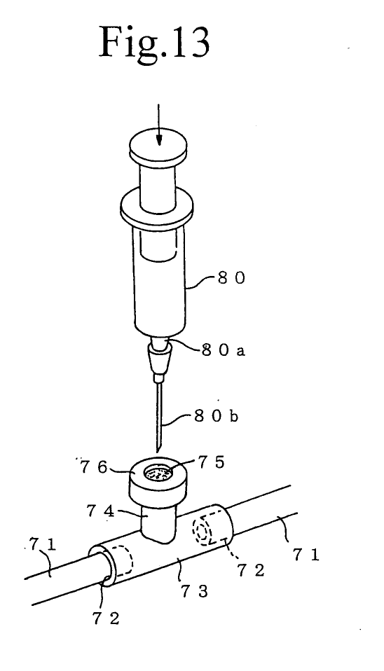

In the following, the operation of the needleless injection adapter 300 according to the third embodiment will be described with reference to FIGS. 11 and 12.

First, both ends of the duct body 52 of the needleless injection adapter 300 according to the third embodiment are connected to the ends of the fluid conveying tubes 68 of an extracorporeal blood circuit (not illustrated). In the needleless injection adapter 300 according to this embodiment, as shown in FIG. 11, the socket 55 of the rotatable member 56 is obliquely biased by the tip end 61a of the spring member 61 at first. In other words, the small hole 58 extending to the socket 55 of the rotatable member 56 is misaligned with the small hole 57 extending through the side face 53 of the duct body 52. In this state, blood flows from the fluid conveying tubes 68 through the duct body 52 without back-flowing through the small holes 57 and 58, so that dialysis treatment and the like are carried out without problem.

When it becomes necessary to inject medical fluid into a patient as shown in FIG. 11, the tip 54a of the injector 54 without a needle is coupled to the socket 55 erected on the rotatable member 56. Then, as shown in FIG. 12, the socket 55 with the injector 54 is rotated against the spring force of the spring member 61 in a vertical direction (in the direction of the arrow). In this case, the tip end 61a of the spring member 61 runs up along the projection 65a on the side face of the socket 55. Falling from the top portion of the projection 65a, the snapped tip end 61a of the spring member 61 emits a sound or an impact (the operation of the sound emission member 65). A time when the sound or impact is emitted, that is, when the sound emission member 65 operates coincides with the time when the small holes 57 and 58 get aligned with each other, so that upon hearing the sound or impact, an operator clearly confirm the alignment of the small holes 57 and 58.

Then, the operator pushes a plunger while vertically holding the socket 55 with the injector 54, to inject medical fluid into the patient through the fluid conveying tubes 68 with blood flowing through the duct body 52. Upon completing this injection operation, the operator loosens his/her vertical hold of the injector 54, allowing it to rotate to be oblique by the spring force of the spring member 61 as shown in FIG. 11. Therefore, the small holes 57 and 58 are brought into the non-communication state. Thereafter, the tip 54a of the injector 54 is pulled out of the socket 55. The patient is connected to the extracorporeal blood circuit (the blood or fluid transfusion set) to continue the original treatment. Accordingly, it is possible to complete a series of medical fluid injection operation with smoothness and with safety.

The needleless injection adapter 300 according to this embodiment is also applied for continuous medical fluid injection of long time by coupling a medical fluid continuous injector (not illustrated) to the socket 55 of the rotatable member 56. Namely, with the alignment of the small hole 58 and the small hole 57, as shown in FIG. 10, the wedge-shaped clearance S is formed between the duct body 52 and the rotatable member 56. Inserting the wedge-shaped member 67 into the clearance S makes it possible to continuously retain the alignment of the small holes 57 and 58.

The invention is not limited to the above embodiments and various modifications may be made without departing from the spirit and scope of the invention. Any improvement may be made in part or all of the components.

1. A needleless injection adapter comprising:
a socket being coupled to a tip of an injector;
a duct body having, at both ends, connecting portions to connect with fluid conveying tubes;
a movable member being slidably held on a middle of a surface of said duct body and having said socket, a first hole, and a second hole, the first hole being formed inside the movable member and extending to said socket, the second hole extending through said duct body, the first and second holes facing to each other on a face on which said movable member and said duct body are slidably in contact with each other; and
a biasing member biasing said movable member in a direction where said first and second holes are misaligned with each other.

2. The needleless injection adapter according to claim 1, wherein,
said biasing member is fixed on said duct body so that said biasing member accumulates return force to be elastically deformed in a state that said socket and an inside of said duct body get in communication with each other by a movement of said movable member.

3. The needleless injection adapter according to claim 1, wherein
said socket has a stopper which maintains the state that said socket is in communication with the inside of said duct body.

4. A needleless injection adapter comprising:
a socket being coupled to a tip of an injector;
a duct body having, at both ends, connecting portions to connect with fluid conveying tubes and having a vertical hole to connect said socket to the inside of said duct body;
a slidable member having said socket and at least one connection hole and dividing said vertical hole connecting said socket to the inside of said duct body, the connection hole connecting upper and lower vertical holes divided by said slidable member; and
a biasing member biasing said slidable member to slide in a direction where said connection hole shifts from the upper and lower vertical holes.

5. The needleless injection adapter according to claim 4, wherein,
said biasing member is fixed on said duct body so that said biasing member accumulates return force to be elastically deformed in a state that said socket and the inside of said duct body get in communication with each other by a sliding of said slidable member.

6. The needleless injection adapter according to claim 4, wherein
said socket has a stopper which maintains the state that said socket is in communication with the inside of said duct body.

7. A needleless injection adapter comprising:
a socket being coupled to a tip of an injector;
a duct body having, at both ends, connecting portions to connect with fluid conveying tubes; and
a rotatable member being slidably held on a middle of a surface of said duct body and having said socket, a first hole, and a second hole, the first hole being formed inside said rotatable member and extending to said socket, the second hole extending through said duct body, the first and second holes facing to each other on a face where said rotatable member and said duct body are slidably in contact with each other;
a spring member biasing said rotatable member in a direction where said first and second holes are misaligned; and
a sound emission member and/or a tactile member being provided in said socket, snapping a tip end of said spring member at a position where said first and second holes are aligned with each other.

8. The needleless injection adapter according to claim 7, wherein
said sound emission member and/or said tactile member include(s) a tip end of said spring member whose base end is held by said duct body; and a projection or a recess on a side face of said socket on which the tip end of said spring member abuts.

9. The needleless injection adapter according to claim 7, further comprising a position retaining member which holds said rotatable member to rotate against a spring force of said spring member, in such a position that said first and second holes are aligned with each other.
